(19) United States
(12) Patent Application Publication
(10) Pub. No.: US 2010/0331349 A1
(43) Pub. Date: Dec. 30, 2010

(54) Use of gamma-aminobutyric acid to mask or reduce an unpleasant flavour impression and preparations containing gamma-aminobutyric

(75) Inventors: Jakob Ley, Holzminden (DE); Gunter Kindel, Hoxter (DE); Gerhard Krammer, Holzminden (DE); Thomas Hofmann, Munster-Roxel (DE); Nina Rotzoll, Hamburg (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Symrise GmbH & Co. KG

(21) Appl. No.: 10/599,701

(22) PCT Filed: Mar. 29, 2005
(86) PCT No.: PCT/EP2005/051424

§ 371 Date: Sep. 10, 2010

(30) Foreign Application Priority Data
Apr. 7, 2004 (DE) 10 2004 017 076.2

(51) Int. Cl. A61K 31/522; A61K 31/439; A61P 43/00; A23L 1/227; A23L 2/56

(52) U.S. Cl. 514/263.34; 514/305; 426/534; 426/442

(57) ABSTRACT

The use of γ-aminobutyric acid or a physiologically acceptable salt of γ-aminobutyric acid to mask or reduce the unpleasant flavour impression of an unpleasantly tasting substance is described. The food, beverage or oral care preparations or oral pharmaceutical preparations comprising at least one unpleasantly tasting substance and γ-aminobutyric acid are also described.

The present invention concerns the masking or reduction of the unpleasant flavour impression of unpleasantly tasting substances, and in particular of substances that impart a bitter, astringent and/or metallic flavour impression. The invention thus concerns (i) processes for masking or reducing such flavour impressions and (ii) food, oral care or beverage preparations or oral pharmaceutics preparations which, despite the presence of one or more substances that usually impart an unpleasant flavour impression, have a pleasant flavour. Like other preparations too, foodstuffs or beverages commonly contain various bitter principles which although on the one hand desirable and characteristic in moderation (e.g. caffeine in tea or coffee, quinine in bitter lemon drinks, hop extracts in beer), can on the other hand also severely detract from the value (e.g. flavonoid glycosides and limonoids in citrus juices, the bitter aftertaste of many artificial sweeteners such as aspartame or saccharine, hydrophobic amino acids and/or peptides in cheese).

In order to reduce a natural content of bitter principles, for example, a subsequent treatment of a preparation is often felt to be necessary, by extraction for example, as in the decaffeination of tea or coffee, or by an enzymatic process, e.g. the treatment of orange juice with a glycosidase to destroy the bitter naringin or the use of special peptidases in the ripening of cheese. Such treatments place a strain on the product, generate waste products and also give rise to solvent residues and other residues (enzymes) in the products, for example.

It is therefore desirable to find substances which can effectively sensorially mask (i.e. reduce to an degree which is no longer perceptible to the senses) or at least reduce unpleasant flavour impressions, in particular bitter, astringent and/or metallic flavour impressions.

The reduction or masking of the bitter taste of many pharmaceutical active ingredients is particularly important, since the willingness of patients, particularly patients who are sensitive to bitter principles, such as children, to take a corresponding preparation orally, can be significantly increased in this way. Many pharmaceutical active ingredients, for example aspirin, salicin, paracetamol, ambroxol or quinine, to name just a very small selection by way of clarification, have a marked bitter, astringent and/or metallic taste and/or aftertaste.

Although some substances are known which can partially suppress a bitter flavour, many of these substances are severely limited in their application.

In U.S. Pat. No. 5,637,618 a bitter taste is reduced using lactisole [2O-(4-methoxyphenyl)lactic acid]. However, this inhibitor also strongly inhibits the sweet flavour impression (cf. U.S. Pat. No. 5,045,336), which severely limits its applicability.

2,4-Dihydroxybenzoic acid potassium salt is described in U.S. Pat. No. 5,643,941 (table column 3, line 18) as a masking agent for the bitter taste of potassium chloride, but it cannot suppress the taste of caffeine, for example.

According to GB 2,380,936 the taste of bitter pharmaceuticals is suppressed with ginger extracts. However, the strong aroma impression and/or the pungency which is commonly to be found in ginger extracts or active ingredients obtained from them is unsuitable for many applications.

Neohesperidin dihydrochalcone likewise has a bitterness-reducing effect, but it is primarily a sweetener (cf. Manufacturing Chemist 2000, July edition, p. 16-17), which also has an intrusive effect in non-sweet applications.

Whilst flavour-modifying properties are described in U.S. Pat. No. 5,580,545 for some flavones (2-phenyl chrom-2-en-4-ones), a bitterness-reducing or suppressing action has not been found.

US 2002 177,576 describes the suppression of a bitter taste by nucleotides, for example cytidine-5′-monophosphates (CMP). The highly polar compounds, which can therefore only be used in highly polar solvents, are only of very limited use in many fatty foodstuffs, however. In addition, the availability of such substances is extremely limited due to their expensive chemical synthesis.

US 2002 188,019 describes hydroxyflavanones as effective masking agents for bitter tastes, but they are only obtainable synthetically with difficulty and are not available in larger amounts at a reasonable cost.

The sodium salts sodium chloride, sodium citrate, sodium acetate and sodium lactate have a bitterness-masking effect against many bitter principles (e.g. Nature, 1997, vol. 387, p. 563); however, the intake of large amounts of sodium ions can lead to heart and circulatory diseases. Disadvantageously, however, a significant bitterness-masking effect only sets in with relatively high sodium concentrations (from about 0.1 M), which corresponds for example to a generally unacceptably high content of about 0.6 wt. % NaCl in the final application (cf. R. S. J. Keast, P. A. S. Breslin and G. K. Beauchamp, Chimia 2001, 55(5), 441-447).

WO 00/21390 describes polyglutamic acid as a bitterness-masking agent; relatively high concentrations of around 1 wt. % are needed in this case.

A lipoprotein consisting of β-lactoglobulin and phosphatidic acid likewise has a bitterness-masking effect (EP-A 635 218). Such polymers are difficult to characterise and standardise, however, and have a pronounced soapy aftertaste.

The flavone glycoside neodiosmin [5,7-dihydroxy-2-(4-methoxy-3-hydroxyphenyl)-7-O-neohesperidosyl chrom-2-en-4-one] likewise has a bitterness-masking effect (U.S. Pat. No. 4,154,862), but it is characterised by a disaccharide radical which makes production or isolation and applicability of the substance much more difficult.

The primary object of the present invention was to find substances which are suitable for masking or reducing the unpleasant flavour impression of unpleasantly tasting substances (and which preferably have in particular a bitterness-masking effect against a large number of bitter principles), can be widely used, occur in nature or in foodstuffs and are easily accessible.

This primary object is achieved by the use of γ-aminobutyric acid (4-aminobutanoic acid) or a physiological acceptable salt of γ-aminobutyric acid to mask or reduce the unpleasant flavour impression of an unpleasantly tasting substance. γ-Aminobutyric acid is particularly suitable as a constituent of food, oral care or beverage preparations and of oral pharmaceutical preparations.

A corresponding process according to the invention to mask or reduce the unpleasant flavour impression of an unpleasantly tasting substance in a food, oral care or beverage preparation comprises the following step:

Mixing an amount of (a) γ-aminobutyric acid (4-aminobutanoic acid; hereinafter also called GABA) or (b) a physiologically acceptable salt of γ-aminobutyric acid with the other constituents of the preparation, the amount being sufficient to sensorially mask or to reduce the unpleasant flavour impression of the unpleasantly tasting substance.

γ-Aminobutyric acid occurs for example in turnips (Beta vulgaris), yeast, the brain, brown rice and green tea (Römpp Lexikon der Naturstoffe, Thieme-Verlag 1997, p. 30) and is also otherwise widespread in plants and animals and in foodstuffs (S.-H. Oh, Y.-J. Moon and C.-H. Oh, Nutraceuticals and Food, 2003, volume 8 no. 1, pages 75-78). It is a neurotransmitter and plays an important role in the transmission of signals between neurones. The use of γ-aminobutyric acid for foodstuffs is not problematic, since humans have always consumed relevant amounts of free γ-aminobutyric acid and no negative physiological effects are known to date.

The acidic taste of γ-aminobutyric acid is known.

JP 2003 159017 (Chemical Abstracts vol. 139, 2003, abstract no. 400860) describes a cereal product in which γ-aminobutyric acid and alanine are enriched through a preparation process, such that the resulting cereal product is, among other things, less bitter than the original cereal. However, the elevated concentration of alanine is made responsible for the effect.

U.S. Pat. No. 4,479,974 describes a method for improving the aroma impression and mouth feel of a beverage preparation, which is achieved inter alia through the addition of 0.1-8% γ-aminobutyric acid. However, the modification is aimed not at masking unpleasant notes but at the mouth feel.

The invention is thus based on the surprising finding that even in very low concentrations of less than 0.1 wt. % in preparations, γ-aminobutyric acid and its physiologically acceptable salts can reduce or even completely suppress the unpleasant flavour impression, in particular the bitter flavour impression, of many substances, in particular of methyl xanthines such as e.g. caffeine, alkaloids such as e.g. quinine, flavonoids such as e.g. naringin, inorganic salts such as potassium chloride or magnesium sulfate, and pharmaceutical active ingredients such as e.g. beta-lactam antibiotics.

It is particularly advantageous in this context that other than a slightly acidic taste γ-aminobutyric acid (and correspondingly its physiologically acceptable salts) has virtually no aroma and does not influence the other, not unpleasant flavour properties of a composition that are generally present.

Unpleasantly tasting substances within the meaning of the present invention are:
(a) Substances which taste bitter, astringent, sticky, dusty, dry, mealy, rancid and/or metallic and
(b) Substances which have a bitter, astringent, sticky, dusty, dry, mealy, rancid or metallic aftertaste.

The abovementioned unpleasantly tasting substances can also have other, generally not unpleasant flavour and/or odour qualities. Examples which can be cited of other, not unpleasant flavour qualities within the meaning of the present invention are, for example, spicy, umami, sweet, salty, sour, sharp, cooling, warming, burning or tingling impressions.

Substances which taste bitter, astringent, sticky, dusty, dry, mealy, rancid or metallic are, for example: xanthine alkaloids, xanthines (caffeine, theobromine, theophylline), alkaloids (quinine, brucine, strychnine, nicotine), phenolic glycosides (e.g. salicin, arbutin), flavonoid glycosides (e.g. hesperidin, naringin), chalcones and chalcone glycosides, hydrolisable tannins (gallic or ellagic acid esters of carbohydrates, e.g. pentagalloyl glucose), non-hydrolysable tannins (optionally galloylised catechins or epicatechins and oligomers thereof, e.g. proanthyocyanidines or procyanidines, thearubigin), flavones (e.g. quercetin, taxifolin, myricetin), other polyphenols (γ-oryzanol, caffeic acid or esters thereof), terpenoid bitter principles (e.g. limonoids such as limonin or nomilin from citrus fruits, lupolones and humolones from hops, iridoids, secoiridoids), absinthin from wormwood, amarogentin from gentian, metallic salts (potassium chloride, sodium sulfate, magnesium sulfate), certain pharmaceutical active ingredients (e.g. fluoroquinolone antibiotics, paracetamol, aspirin, beta-lactam antibiotics, ambroxol, propyl thiouracil [PROP], guaifenesin), certain vitamins (for example vitamin H, B-series vitamins such as vitamin B1, B2, B6, B12, niacin, panthotenic acid), denatonium benzoate, sucralose octaacetate, potassium chloride, magnesium salts, iron salts, aluminium salts, zinc salts, urea, unsaturated fatty acids, in particular unsaturated fatty acids in emulsions, amino acids (e.g. leucine, isoleucine, valine, tryptophane, proline, histidine, tyrosine, lysine and phenylalanine), peptides (in particular peptides with an amino acid from the group comprising leucine, isoleucine, valine, tryptophane, proline or phenylalanine at the N- or C-terminus).

Substances which have a bitter, astringent, sticky, dusty, dry, mealy, rancid or metallic aftertaste can belong for example to the group of sweeteners or sugar substitutes. Examples which can be cited include aspartame, neotame, superaspartame, saccharine, sucralose, tagatose, monellin, stevioside, thaumatin, miraculin, glycerrhizin and derivatives thereof, cyclamate and the pharmaceutically acceptable salts of the abovementioned compounds.

A further aspect of the invention, which is closely associated with the use according to the invention of γ-aminobutyric acid, concerns preparations. Preparations according to the invention are used for (a) foodstuffs, (b) beverages or (c) oral care or are (d) oral pharmaceutical preparations or are (e) cosmetic preparations for application in the head region. They comprise:
at least one unpleasantly tasting substance and
γ-aminobutyric acid
   wherein
   the amount of the unpleasantly tasting substance is sufficient to be perceived as an unpleasant taste in a comparative preparation which contains no γ-aminobutyric acid but otherwise has an identical composition, and
   the amount of γ-aminobutyric acid is sufficient to sensorially mask the unpleasant flavour impression of the unpleasantly tasting substance or to reduce it in comparison with the comparative preparation.

By starting from a comparative preparation which contains a perceptible (tastable) amount of an unpleasantly tasting substance and adding to it an amount of γ-aminobutyric acid which is sufficient to sensorially mask the unpleasant flavour impression of the unpleasantly tasting substance (i.e. to reduce it to a degree which is no longer perceptible to the senses) or to reduce it in comparison with the comparative preparation, a preparation according to the invention is thus obtained.

Preferred preparations according to the invention are oral care preparations which in addition to the aforementioned constituents contain one or more oral care substances in an amount which is effective for oral care.

Preparations according to the invention, e.g. oral care preparations, containing 0.000001 to 0.1 wt. % of (a) γ-aminobutyric acid or (b) physiologically acceptable salts of γ-aminobutyric acid, based on the total weight of the preparation, are particularly preferred.

Preparations according to the invention may but do not have to be in the form of a finished product. Particularly preferred preparations are in the form of a semi-finished product, a perfume, aromatic or flavouring composition or a spice mix.

Preparations according to the invention can contain, in addition to (a) γ-aminobutyric acid or (b) physiologically acceptable salts of γ-aminobutyric acid, at least one further substance (flavour corrective) to modify, mask or reduce the unpleasant flavour impression of an unpleasantly tasting substance. This can be useful in particular for treating certain combinations of unpleasantly tasting substances highly efficiently.

Food or beverage preparations according to the invention are, for example, baked goods (e.g. bread, dry biscuits, cakes, other pastries), confectionery (e.g. chocolates, chocolate bar products, other bar products, fruit gums, hard and soft caramels, chewing gum), alcoholic or non-alcoholic drinks (e.g. coffee, tea, wine, wine-based drinks, beer, beer-based drinks, liqueurs, spirits, brandies, fruit-based soft drinks, isotonic drinks, soft drinks, nectars, fruit and vegetable juices, fruit or vegetable juice preparations), instant drinks (e.g. instant chocolate drinks, instant tea drinks, instant coffee drinks), meat products (e.g. ham, cured or uncured sausage preparations, spiced or marinated fresh or salted meat products), eggs or egg products (dried egg, egg white, egg yolk), cereal products (e.g. breakfast cereals, muesli bars, pre-fermented prepared rice products), dairy products (e.g. milk drinks, ice cream, yoghurt, kefir, cream cheese, soft cheese, hard cheese, dried milk powder, whey, butter, buttermilk), products made from soya protein or other soya bean fractions (e.g. soya milk and products made therefrom, preparations containing soya lecithin, fermented products such as tofu or tempe or products made therefrom), fruit preparations (e.g. jams, fruit sorbets, fruit sauces, fruit fillings), vegetable preparations (e.g. ketchup, sauces, dried vegetables, frozen vegetables, pre-fermented vegetables, preserved vegetables), snacks (e.g. baked or fried potato crisps or potato dough products, extruded products based on maize or peanuts), products based on fats and oils or emulsions thereof (e.g. mayonnaise, remoulade, dressings), other ready meals and soups (e.g. dried soups, instant soups, pre-fermented soups), spices, spice mixes and in particular seasonings, which are used in the snacks sector for example.

The preparations according to the invention can also be used as semi-finished products for the production of other food or beverage preparations, for example. The preparations according to the invention can also take the form of capsules, tablets (uncoated and coated tablets, e.g. stomach acid-resistant coatings), pastilles, granules, pellets, solids mixtures, dispersions in liquid phases, emulsions, powders, solutions, pastes or other swallowable or chewable preparations as food supplements.

Oral care (oral hygiene) preparations according to the invention are in particular tooth care products (such as toothpastes, tooth gels, tooth powders), mouthwashes, chewing gums and other oral care products.

Oral pharmaceutical preparations according to the invention are preparations which take the form for example of capsules, tablets (uncoated and coated tablets, e.g. stomach acid-resistant coatings), pastilles, granules, pellets, solids mixtures, dispersions in liquid phases, emulsions, powders, solutions, pastes or other swallowable or chewable preparations and which are used as prescription drugs, over-the-counter drugs or other drugs or as food supplements.

The γ-aminobutyric acid can be used (a) in a neutral form (“inner salt”), (b) in the carboxylate or (c) in the ammonium form, wherein corresponding cations or anions are present as counterions. Use in a neutral form is preferred because of the good availability and formulatability.

In case (b), the unipositively charged cations from the first main and subgroup, the ammonium ion, the trialkyl ammonium ion, the divalently charged cations from the second main and subgroup and the trivalent cations from the third main and subgroup can be used as cations, preferably Na+, K+, NH4+, Ca2+, Mg2+, Al3+ and Zn2+. If sodium ions are used as cations, a two-fold action can occur, since Na+ itself has a masking effect; in that case, however, the presence of the anion of γ-aminobutyric acid is usually critical, at least with a sodium ion concentration of less than 0.1 M (see above); the use of the sodium salt of GABA in a concentration of less than 0.1 M is usually sufficient for a good masking result. In case (c), the uninegatively or multinegatively charged anions of the halides and complex inorganic acids, e.g. of sulfuric acid, phosphoric acid, carbonic acid or pyrophosphoric acid or organic carboxylic acids, preferably the natural alkanoic, hydroxyalkanoic, sugar and fruit acids, can be used as anions, particularly preferably chloride, hydrogen sulfate, sulfate, phosphate, hydrogen phosphate, dihydrogen phosphate, carbonate, hydrogen carbonate, pyruvate, lactate, citrate, tartrate, oxalate, maleate, acetate, propionate or glucorunate anions.

The various salts of γ-aminobutyric acid can naturally be used (i) alone or (ii) as mixtures.

In particularly preferred preparations according to the invention γ-aminobutyric acid or its physiologically acceptable salts are used in combination with one or more flavour correctives. A particularly effective masking can be obtained in this way. In particular, the combination of γ-aminobutyric acid or its physiologically acceptable salts with another flavour corrective for unpleasant, in particular bitter flavour impressions is effective.

Other flavour correctives can be selected from the following list, for example: nucleotides (e.g. adenosine-5′-monophosphate, cytidine-5′-monophosphate), lactisole, sodium salts, hydroxyflavanones, or mixtures of whey proteins with lecithins.

It has already been mentioned that particularly preferred preparations according to the invention, e.g. oral care preparations, contain 0.000001 to 0.1 wt. % of (a) γ-aminobutyric acid or (b) physiologically acceptable salts of γ-aminobutyric acid, based on the total weight of the preparation. Other conventional active ingredients, basic substances, auxiliary substances and additives for foodstuffs, oral care products or beverages or oral pharmaceutical preparations are conventionally included in quantities of 5 to 99.999999 wt. %, preferably 10 to 80 wt. %, based on the total weight of the preparation according to the invention. The preparations according to the invention can also contain water in a quantity of up to 99.999999 wt. %, but preferably in the range from 5 to 80 wt. %, based on the total weight of the preparation.

According to a first preferred embodiment, the preparations according to the invention containing γ-aminobutyric acid or its physiologically acceptable salts are produced by incorporating γ-aminobutyric acid or its physiologically acceptable salts without solvent, as a solution or in the form of a mixture with a solid or liquid carrier in a food, oral care or beverage base preparation (“base”, i.e. containing no GABA or only so little GABA that an unpleasant flavour is not sensorially masked or reduced) or in an oral pharmaceutical base preparation. Preparations according to the invention in the form of a solution can advantageously also be converted to a solid preparation according to the invention by spray drying.

According to a further preferred embodiment, in order to produce preparations according to the invention, γ-aminobutyric acid or its physiologically acceptable salts and optionally other constituents of the preparation according to the invention are first incorporated into emulsions, liposomes, e.g. starting from phosphatidyl cholin, microspheres, nanospheres or into capsules, granules or extruded products made from a suitable matrix for foodstuffs and beverages, e.g. from starch, starch derivatives, cellulose or cellulose derivatives (e.g. hydroxypropyl cellulose), other polysaccharides (e.g. alginate), natural fats, natural waxes (e.g. beeswax, carnauba wax) or from proteins, e.g. gelatine. A preparation according to the invention is particularly preferred wherein the matrix is chosen such that γ-aminobutyric acid or its physiologically acceptable salts undergo a delayed release from the matrix, such that a lasting effect is achieved.

In a further preferred production process, γ-aminobutyric acid or its physiologically acceptable salts are first complexed with one or more suitable complexing agents, for example with cyclodextrins or cyclodextrin derivatives, preferably beta-cyclodextrin, and used in this complexed form.

As constituents for food or beverage preparations according to the invention, the conventional basic substances, auxiliary substances and additives for foodstuffs or beverages can be used, e.g. water, mixtures of fresh or processed, plant-based or animal-based basic substances or raw materials (e.g. raw, roast, dried, fermented, smoked and/or boiled meat, bone, gristle, fish, vegetables, fruit, herbs, nuts, vegetable or fruit juices or pastes or mixtures thereof), digestible or indigestible carbohydrates (e.g. sucrose, maltose, fructose, glucose, dextrin, amylose, amylopectin, inulin, xylan, cellulose), sugar alcohols (e.g. sorbitol), natural or hydrogenated fats (e.g. tallow, lard, palm oil, coconut butter, hydrogenated vegetable fat), oils (e.g. sunflower oil, groundnut oil, maize oil, olive oil, fish oil, soya oil, sesame oil), fatty acids or salts thereof (e.g. potassium stearate), proteinogenic or non-proteinogenic amino acids and related compounds (e.g. taurin), peptides, native or processed proteins (e.g. gelatine), enzymes (e.g. peptidases), nucleic acids, nucleotides, flavour correctives for unpleasant flavour impressions, flavour correctives for other, generally not unpleasant flavour impressions, flavour-modulating substances, e.g. inositol phosphate, nucleotides such as guanosine monophosphate, adenosine monophosphate or other substances such as sodium glutamate or 2-phenoxypropionic acid), emulsifiers (e.g. lecithins, diacyl glycerols), stabilisers (e.g. carageenan, alginate), preservatives (e.g. benzoic acid, sorbic acid), antioxidants (e.g. tocopherol, ascorbic acid), chelating agents (e.g. citric acid), organic or inorganic acidulators (e.g. malic acid, acetic acid, citric acid, tannic acid, phosphoric acid), additional bitter principles (e.g. quinine, caffeine, limonin, amarogentin, humolones, lupolones, catechins, tannins), sweeteners (e.g. saccharine, cyclamate, aspartame, neotame), mineral salts (e.g. sodium chloride, potassium chloride, magnesium chloride, sodium phosphate), substances preventing enzymatic browning (e.g. sulfite, ascorbic acid), essential oils, plant extracts, natural or synthetic dyes or coloured pigments (e.g. carotinoids, flavonoids, anthocyans, chlorophyll and derivatives thereof), herbs, trigeminally active substances or plant extracts containing trigeminally active substances, synthetic, natural or nature-identical aromatic substances or perfumes and odour correctives.

Tooth care products (as an example of oral care preparations), which contain γ-aminobutyric acid or its physiologically acceptable salts, generally contain an abrasive system (grinding or polishing agent), such as e.g. silicas, calcium carbonates, calcium phosphates, aluminium oxides and/or hydroxyl apatites, surface-active substances, such as e.g. sodium lauryl sulfate, sodium lauryl sarcosinate and/or cocamidopropyl betaine, humectants, such as e.g. glycerol and/or sorbitol, thickeners, such as e.g. carboxymethyl cellulose, polyethylene glycols, carrageenans and/or Laponites®, sweeteners, such as e.g. saccharine, flavour correctives for unpleasant flavour impressions, flavour correctives for other, generally not unpleasant flavour impressions, flavour-modulating substances (e.g. inositol phosphate, nucleotides such as guanosine monophosphate, adenosine monophosphate or other substances such as sodium glutamate or 2-phenoxypropionic acid), cooling agents, such as e.g. menthol or menthol derivatives, stabilisers and active ingredients, such as e.g. sodium fluoride, sodium monofluorophosphate, tin difluoride, quatenary ammonium fluorides, zinc citrate, zinc sulfate, tin pyrophosphate, tin dichloride, mixtures of various pyrophosphates, triclosan, cetyl pyridinium chloride, aluminium lactate, potassium citrate, potassium nitrate, potassium chloride, strontium chloride, hydrogen peroxide, aromas and/or sodium bicarbonate or odour correctives.

Chewing gums (as a further example of oral care preparations), which contain γ-aminobutyric acid or its physiologically acceptable salts, generally comprise a chewing gum base, in other words a chewing compound that becomes plastic when chewed, sugars of various types, sugar substitutes, sweeteners, sugar alcohols, flavour correctives for unpleasant flavour impressions, flavour correctives for other, generally not unpleasant flavour impressions, flavour-modulating substances (e.g. inositol phosphate, nucleotides such as guanosine monophosphate, adenosine monophosphate or other substances such as sodium glutamate or 2-phenoxypropionic acid), humectants, thickeners, emulsifiers, aromas and stabilisers or odour correctives.

All conventionally used active ingredients, basic substances, auxiliary substances and additives for oral pharmaceutical preparations can be used as constituents for oral pharmaceutical preparations according to the invention. In particular, unpleasantly tasting orally formulatable pharmaceutical active ingredients can also be used as active ingredients. The active ingredients, basic substances, auxiliary substances and additives can be converted to the oral administration forms by methods known per se. This is commonly done using inert, non-toxic, pharmaceutically suitable auxiliary substances. These include inter alia carriers (e.g. microcrystalline cellulose), solvents (e.g. liquid polyethylene glycols), emulsifiers (e.g. sodium dodecyl sulfate), dispersants (e.g. polyvinyl pyrrolidone), synthetic and natural biopolymers (e.g. albumin), stabilisers (e.g. antioxidants such as ascorbic acid), dyes (e.g. inorganic pigments such as iron oxides) or odour correctives and flavour correctives not affecting the bitter flavour.

The preparations according to the invention can preferably also contain an aromatic composition to round off and to improve the flavour and/or smell of the preparation. Suitable aroma compositions include, for example, synthetic, natural or nature-identical aromatic, perfume and flavouring substances and suitable auxiliary substances and carriers. It is regarded as being particularly advantageous if a bitter or metallic flavour impression deriving from aromatic substances or perfumes contained in the preparations according to the invention can be masked or reduced, thereby improving the overall aroma or flavour profile of the preparation.

Preparations according to the invention in the form of semi-finished products can be used to mask or reduce the unpleasant flavour impression of finished preparations produced using the semi-finished preparation.

Preparations according to the invention which are used as semi-finished products generally contain 0.0001 wt. % to 95 wt. %, preferably 0.001 to 80 wt. %, but in particular 0.01 wt. % to 50 wt. %, based on the total weight of the preparation, of γ-aminobutyric acid or its physiologically acceptable salts and one or more perfumes and aromatic substances, optionally also various carriers and auxiliary substances or various solvents. Also particularly preferred are semi-finished products in the form of emulsions, liposomes, microspheres, nanospheres or capsules, spray-dried products, granules or extruded products made from a suitable matrix for foodstuffs and beverages, e.g. starch, starch derivatives, cellulose or cellulose derivatives (e.g. hydroxypropyl cellulose), other polysaccharides (e.g. alginate), natural fats, natural waxes (e.g. beeswax, carnauba wax) or proteins, e.g. gelatine.

Finally the invention also concerns (i) the use of γ-aminobutyric acid or its physiologically acceptable salts in cosmetic preparations to mask or reduce the unpleasant flavour impression of an unpleasantly tasting substance and (ii) the corresponding cosmetic preparations (formulations) themselves, and in particular those which contain an unpleasantly tasting substance and which even when applied correctly to the skin can come into contact with the oral cavity, in other words, for example—as already mentioned—cosmetic preparations for application in the head area, such as soaps, other cleansing or care products for the facial area, face creams or lotions or ointments, sunscreens, beard shampoos or conditioners, shaving foams, soaps or gels, lipsticks or other lip cosmetics or lip care products.

### EXAMPLES

The examples below serve to clarify the invention.

### Application Example 1

### Bitterness Reduction in a Caffeine Solution

To quantify the reduction in the bitter impression with the use of γ-aminobutyric acid, the bitterness of a 500 ppm caffeine solution (comparative solution; not according to the invention) and of four samples according to the invention containing 500 ppm of caffeine and differing amounts of γ-aminobutyric acid was determined by a group of experts (rating 0 [not bitter] to 10 [extremely bitter]).

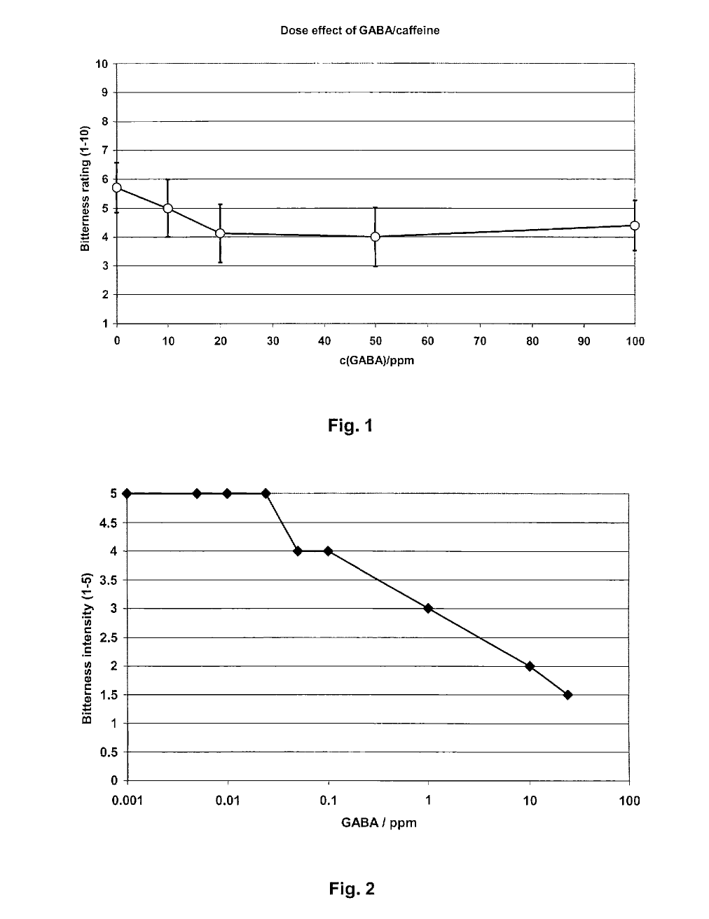

FIG. 1 shows the relative reduction in the bitterness of solutions containing 500 ppm of caffeine and varying amounts of γ-aminobutyric acid (GABA for short) in comparison with a 500 ppm caffeine solution (without GABA).

### Application Example 2

### Bitterness Reduction in a Quinine Solution

To quantify the reduction in the bitter impression, the bitterness of a 12.5 ppm quinine hydrochloride solution (comparative solution; not according to the invention) and of eight samples according to the invention containing 12.5 ppm of quinine hydrochloride and differing amounts of γ-aminobutyric acid was determined by a group of experts (rating 0 [not bitter] to 5 [extremely bitter]).

FIG. 2 shows the relative reduction in the bitterness of solutions containing 12.5 ppm of quinine hydrochloride and varying amounts of γ-aminobutyric acid (GABA for short) in comparison with a solution containing 12.5 ppm of quinine hydrochloride solution (but no GABA).

### Application Example 3

### Combination of Sodium Salt of Homoeriodictyol with γ-Aminobutyric Acid

To quantify the reduction in the bitter impression, the bitterness of (a) a 500 ppm caffeine solution (basic solution), (b) a sample containing 500 ppm of caffeine, 100 ppm of homoeriodictyol sodium salt and 20 ppm of γ-aminobutyric acid, and (c) a sample containing 500 ppm of caffeine and 20 ppm of γ-aminobutyric acid was determined by a group of experts (rating 1 [not bitter] to 10 [extremely bitter]). The evaluation was made as a calculation of the reduction (in %) of the bitter impression from the average values for the ratings of the caffeine solution and the solutions containing caffeine and γ-aminobutyric acid.

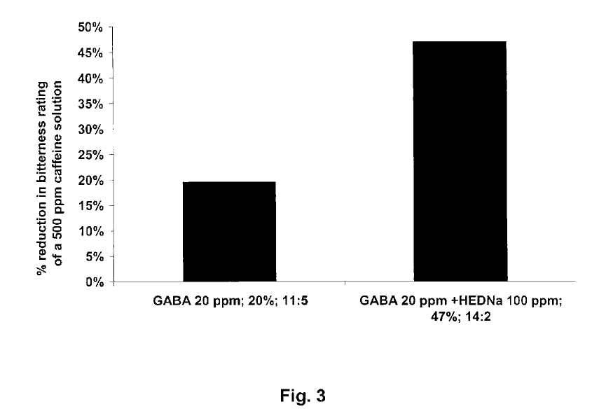

FIG. 3 shows the relative reduction in the bitterness of a solution containing 500 ppm of caffeine by the addition of 20 ppm of γ-aminobutyric acid (GABA for short) (left) and 20 ppm GABA and 100 ppm of homoeriodictyol sodium salt (HEDNa) (right).

### Application Example 4

### Use in a Soya Drink

γ-Aminobutyric acid was predissolved in water and added to a soya milk from a local supermarket. The mixture was mixed with the milk aroma in a beaker.

| | |
|---|---|
| Sample 1 | Soya milk (local supermarket) |
| Sample 2 | Sample 1 + 10 ppm γ-aminobutyric acid + 0.1% milk aroma |

The profile was produced by a panel of 4 experts by description using predefined descriptors.

| | |
|---|---|
| Sample 1 | green, bean, fatty (oily), rancid, nut (hazelnut), sweet, very |
| | astringent, very dry/dusty, mealy, sticky, cereal (bran), |
| | adhesive, false note |
| Sample 2 | A little fresher, fatty, nutty notes are masked, top note is |
| | moderated, not quite so dry and sticky |

### Application Example 5

### Use in Combination in a Soya Drink

γ-Aminobutyric acid and homoeriodictyol sodium salt were predissolved in water and added to a soya milk from a local supermarket. The mixture was mixed with the milk aroma in a beaker.

| | |
|---|---|
| Sample 1 | Soya milk (local supermarket) |
| Sample 2 | Sample 1 + 10 ppm γ-aminobutyric acid + 100 ppm |
| | homoeriodictyol sodium salt + 0.1% milk aroma |

The profile was produced by a panel of 4 experts by description using predefined descriptors.

| | |
|---|---|
| Sample 1 | green, bean, fatty (oily), rancid, nut (hazelnut), sweet, very |
| | astringent, very dry/dusty, mealy, sticky, cereal (bran), |
| | adhesive, false note |
| Sample 2 | slightly vanilla, soft, not so sticky and dry, sweeter, slightly |
| | different character |

1. A process for masking unpleasant tastes and/or odors in a preparation by adding γ-aminobutyric acid or a physiologically acceptable salt of γ-aminobutyric acid in an amount sufficient to mask or reduce the unpleasant flavour impression of an unpleasantly tasting substance.

2. Process to mask or reduce the unpleasant flavour impression of an unpleasantly tasting substance in a food, oral care or beverage preparation or oral pharmaceutical preparation, comprising the following step:
mixing an amount of (a) γ-aminobutyric acid or (b) a physiologically acceptable salt of γ-aminobutyric acid with the other constituents of the preparation, the amount being sufficient to sensorially mask or to reduce the unpleasant flavour impression of the unpleasantly tasting substance.

3. A preparation useful as a food, beverage, oral care composition, oral pharmaceutical composition, or cosmetic composition, for application in the head region, said preparation comprising:
at least one unpleasantly tasting substance and
γ-aminobutyric acid
wherein
the amount of the unpleasantly tasting substance is sufficient to be perceived as an unpleasant taste in a comparative preparation which contains no γ-aminobutyric acid but otherwise has an identical composition, and the amount of γ-aminobutyric acid is sufficient to sensorially mask the unpleasant flavour impression of the unpleasantly tasting substance or to reduce it in comparison with the comparative preparation.

4. A preparation according to claim 3, further comprising:
one or more oral care substances in an amount which is effective for oral care.

5. Preparation according to claim 3, in the form of a semi-finished product, a perfume, an aromatic composition, a flavouring composition or a spice mix.

6. Preparation according to claim 3 useful as a cosmetic and selected from the group comprising: soaps, cleansing and care products for the facial region, face creams, face lotions, facial ointments, sunscreens, beard shampoos, beard conditioners, shaving foams, shaving soaps, shaving gels, lipsticks, lip care products and lip cosmetics.

7. Preparation according to claim 3, comprising 0.000001 to 0.1 wt. % of (a) γ-aminobutyric acid or (b) physiologically acceptable salts of γ-aminobutyric acid, based on the total weight of the preparation.

8. Preparation according to claim 3, further comprising:
at least one further substance for modifying, masking or reducing the unpleasant flavour impression of an unpleasantly tasting substance.
